(19) United States
(12) Patent Application Publication
(10) Pub. No.: US 2012/0273358 A1
(43) Pub. Date: Nov. 1, 2012

(54) METHOD OF TREATMENT OF CELLULOSIC OBJECTS

(75) Inventors: Erik Larnoy, Nesoddtangen (NO); Andreas Treu, Oslo (NO); Hasse V. Halvorsen, Porsgrunn (NO)

(73) Assignee: MILJOTEKNOLOGI AS, Porsgrunn (NO)

(21) Appl. No.: 13/504,382

(22) PCT Filed: Oct. 28, 2010
(86) PCT No.: PCT/GB10/02005

§ 371 Date: Jul. 9, 2012

(30) Foreign Application Priority Data
Oct. 28, 2009 (GB) 0918940.8

(51) Int. Cl. A61L 2/03; A23K 3/00

(52) U.S. Cl. 204/551; 204/600

(57) ABSTRACT

The invention provides a method of inhibiting biological damage to a cellulosic structural element exposed to environmental moisture, said method comprising applying a pulsed asymmetric electrical voltage to a decay-prone region of a said element over a prolonged time period.

This application is entitled to the benefit of, and incorporates by reference essential subject matter disclosed in PCT Application No. PCT/GB2010/002005 filed on Oct. 28, 2010, which claims priority to Great Britain Application No. 0918940.8 filed Oct. 28, 2009.

### BACKGROUND OF THE INVENTION

1. Technical Field

This invention relates to a method of treatment of cellulosic products to inhibit parasitic biological damage, especially fungal or insect damage, and to apparatus therefor.

2. Background Information

Cellulosic items, especially of wood or cloth, when exposed to the environment, are subject to biological deterioration, e.g. staining, decay, or other damage, especially fungal decay. The most commonly-used European timber for products with exterior applications, e.g. house cladding, fences, floors, window frames, doors, noise-barriers, etc, has little inherent resistance to fungal decay as compared to the more durable tropical hard woods. Durability is thus generally enhanced by treatment with chemical wood preservatives, biocides, such as arsenic, chromium or copper-based chemicals or creosote or by using modified wood. Such chemical products however may represent potential environmental pollutants or may negatively affect the visual appearance of the wood. There is thus a need for environmentally tolerable and economically viable alternatives to current chemical wood preservatives.

Besides damage by fungus wood is also subject to damage by insects or insect larvae, in particular termites and beetles, especially Death Watch beetle and ground termites (Rhinoteriniditae).

We have now surprisingly found that the biological deterioration, e.g. fungal decay or staining, of cellulosic material and insect damage to wood may be inhibited by application of an electrical voltage, in particular application of pulsed electrical voltages, and especially DC voltages, at levels which are economically feasible, safe and do not damage the structural integrity of the wood or structures formed from it.

### SUMMARY OF THE DISCLOSURE

Thus viewed from one aspect the invention provides a method of inhibiting biological (e.g. fungal or insect) damage to a cellulosic structural element or an animal feed exposed to environmental moisture, said method comprising applying a pulsed asymmetric electrical voltage to a decay-prone region of a said element or feed over a prolonged time period.

Viewed from a further aspect the invention provides a cellulosic structural element incorporating apparatus for inhibiting biological (e.g. fungal or insect) damage thereof, said apparatus comprising a source of a pulsed asymmetric electrical voltage electrically connected to a decay-prone region of said element.

Viewed from a still further aspect the invention provides apparatus for inhibiting biological (e.g. fungal or insect) damage to a cellulosic structural element, said apparatus comprising a voltage modulator (e.g. a rectifier or transformer) connectable to an electric voltage source and connected via an electric lead to a cellulosic element-engaging electrode.

In the method of the invention, any convenient electrical power source may be used, for example a battery, a solar cell or mains electricity. The voltage applied to the structural element or feed may be derived from a DC (direct current) voltage or an AC (alternating current) source, as for example domestic mains electricity. Pulsed electrical voltages may be achieved in conventional fashion. As an alternative to pulsed DC voltage, stepped DC voltage or asymmetric AC voltage may be used, e.g. by the application of AC voltage with pulses of one polarity being longer or of greater intensity or by the application of DC voltage in alternating steps of different voltages. Pulsed asymmetric treatment in this fashion is often referred to as electroosmotic treatment since it may serve to drive water out of a porous substrate such as concrete or the like. However the damage protective effect of the method of the invention does not rely upon the drying of the cellulosic material by electroosmosis.

The voltage applied will typically be less than 300V, especially 120V or lower, e.g. 5 to 50V. The current within the treated region of the structural element will typically be less than 1 Amp, especially less than 1 mA, particularly less than 0.1 mA, e.g. 0.01 to 0.1 mA. This ensures effective treatment without risk to humans or animals coming into contact with the structural element or feed.

Treatment may be continuous or may be effected only when the structural element or feed has been exposed to moisture, e.g. after rainfall, during spring, autumn or winter, etc. In general, treatment will be for at least 24 hours, preferably at least 1 week, more preferably at least 6 weeks, especially at least 12 weeks, repeated or continued for as long as desired or necessary. During periods of treatment, the voltage application may of course be temporarily halted, generally for a total of no more than 10% of the treatment period, e.g. to allow access or repair.

The voltage may be applied by contacting the structural element or feed with two or more electrodes and/or by relying on moist wood or feed to earth itself. The electrodes may be placed on the surface of the cellulosic element or feed or, more preferably, they may penetrate into the cellulosic element or feed. Spiked electrodes which may be driven into the wood element or feed are especially preferred. Electrical leads to the electrodes are preferably insulated.

The decay-prone regions of the cellulosic element or feed will generally be regions within or in contact with the ground or water, under surfaces, facing poorly ventilated air spaces, internal corners or joints, e.g. below an overhang, poorly drained surfaces (e.g. water-traps), and the like. Regions prone to cyclic wetting are particularly vulnerable. Where these regions do not allow for natural earthing, the electrodes will preferably be such as to close the electrical circuit to the source or to earth the decay-prone region.

The cellulosic element may be of wood as such, especially soft wood, in particular pine or spruce. However the method is also applicable to hard woods such as beech. Besides wood as such, the structural element may be of wood- or plant-derived material, i.e. cellulosic material, such as for example chipboard, plywood, cloth, wood-plastics composites, etc, which are frequently used in interior or exterior applications in moist places, e.g. kitchens, doors, windows, bathrooms, basements, garages, awnings, etc. The structural element may take any convenient form, e.g. sheet, pole, plank, etc, such as described above.

Treatment of the structural element is preferably commenced on installation of the element and continued for its operative lifetime. However already installed structural elements may also be treated according to the invention, preferably before significant fungal or insect damage has occurred. Thus the treatment according to the invention may serve both to reduce further damage to damaged material or to inhibit damage of undamaged material.

The element moreover may be a living tree, e.g. the trees in a plantation. In this event, electrical power is preferably provided by a solar panel.

Besides cellulosic cloths, the method of the invention is also applicable to plastics cloths where fungal infestation manifests itself in discoloration rather than decay of the cloth itself.

Where the structural element is or is to be earthed, this is preferably via a connection which has lower electrical resistance under moist conditions than under dry conditions as in this way the overall current consumption is reduced and economy of the method is improved. To this end, earthing may for example be via surface water on the structural element during rainfall or via a porous non-conducting lead which when moist becomes conducting.

In one embodiment of the invention, the treated substrate may be an animal feed, e.g. pelletized fish or mammal feed, e.g. sweet feed, grain, crushed seed, hay or straw, or animal bedding, e.g. straw. Such feed and bedding is conventionally stored in plastic bags or wrapped in plastic, which if perforated during transport or storage may allow water to enter and cause contamination by fungal growth. This is particularly detrimental with fish feed and feed for horses. In this embodiment, electrodes may conveniently be inserted into the feed through the plastic packaging. To minimize unwanted damage to the feed, such electrodes may be mounted on adhesive holders to provide a water-tight seal at the point of penetration of the packaging. Alternatively, feed packages may be stored on support bases provided with spike electrodes that penetrate into the feed when the feed packages are placed upon the support bases.

Active electrodes, and optionally counter-electrodes to earth the structural element or complete the circuit to the electrical source, may conveniently be disposed in arrays or grids on a non-exposed side of the structural element, e.g. the underside of decking planks, the inner side of house cladding, or as wires interwoven within a cloth structure which is to be treated, etc. In this way the visual appearance of the structural element is unaffected.

The treatment according to the invention may of course be combined with chemical treatment of a cellulosic element, for example with metal-based biocides—however in this event it may be desirable to use lower than conventional dosages. Where conventional chemical treatment is effected, the treatment according to the invention may be limited to areas of particularly high risk of fungal damage, e.g. areas which are embedded in the ground, close to the ground surface, or poorly ventilated or drained. Where the electric treatment is asymmetric, e.g. as in electroosmotic treatment, the electric field applied may be used to drive cellulose-protecting ions, e.g. metal ions, into the cellulosic material to enhance its resistance to damage, especially fungal damage.

While the method of the invention is particularly applicable to the inhibition of fungal damage, it is also particularly applicable to inhibition of insect damage, especially where, as with termites, damage is exacerbated by monocellular symbionts.

Electropulse treatment has been proposed for drying out of inorganic porous components of buildings and the invention is particularly applicable to buildings or other structures with electropulse drying apparatus as the same electrical source may also be used for treatment of cellulosic structural elements in, on or near such buildings. Such electropulse treatment is described for example in U.S. Pat. No. 5,755,945, the contents of which are hereby incorporated by reference.

By delaying or preventing fungal decay or growth, the method of the invention prolongs the period of carbon sequestration by the cellulosic element so further contributing benefit to the environment.

### BRIEF DESCRIPTION OF THE DRAWINGS

Embodiments of the invention will now be described further with reference to the following non-limiting examples and to the accompanying drawings, in which:

FIG. 1 is a schematic diagram of a first apparatus according to the invention;

FIG. 2 is a schematic diagram of a second apparatus according to the invention; and

FIG. 3 is a graph showing mass loss (i.e. decay) of wood samples which are exposed to brown rot fungus and either treated according to the invention or not.

### DETAILED DESCRIPTION OF THE INVENTION

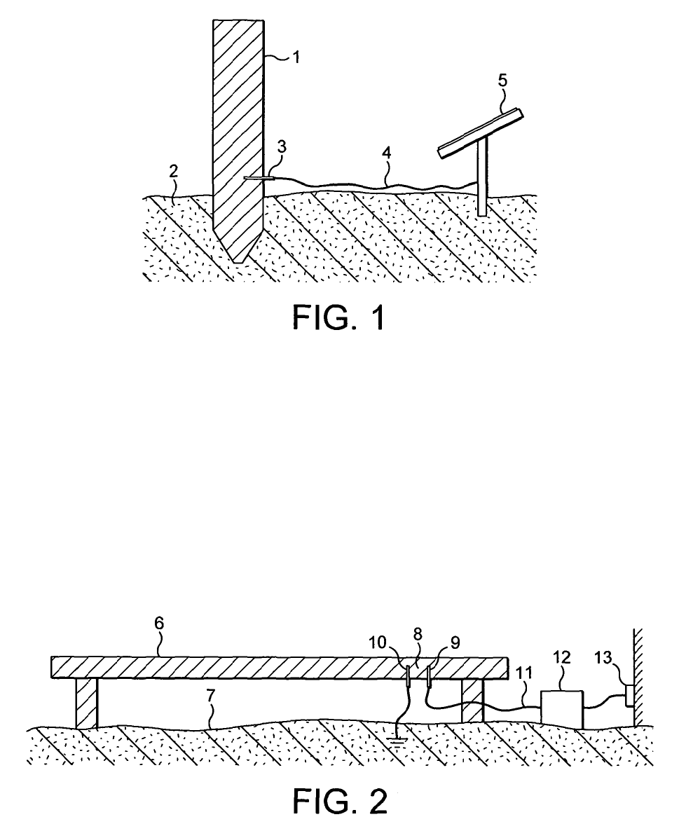

Referring to FIG. 1 there is shown a wooden fence post 1 embedded in soil 2. An electrode spike 3 is embedded in the base of fence post 1, just above ground level. Spike 3 is connected via electrical lead 4 to a solar panel 5 which acts as the electrical power source.

Referring to FIG. 2 there is shown a horizontal plank 6 of a garden deck suspended above ground surface 7. At a decay-prone zone 8 of the plank, the plank is penetrated by active electrode 9 and earthed electrode 10. The active electrode 9 is connected via lead 11 and rectifier/transformer 12 to the domestic electricity supply

### EXAMPLE 1

5 mm×10 mm×30 mm samples of sapwood of Scots pine (Pinus sylvestris) were placed in Petri dishes containing a wood-destroying fungus, Coniophora puteana (brown rot fungus), and a culture medium. Certain samples were connected via 1 kΩ or 11.5 kΩ resistors to a 40V voltage DC source. The samples were incubated for 8 weeks. The weight loss was then determined and found to be negligible. For wood treated with conventional chemical preservatives under these conditions the weight loss should be less than 3%. For untreated wood under these conditions the fungally caused weight loss is much higher, reaching over 20% after 16 weeks.

### EXAMPLE 2

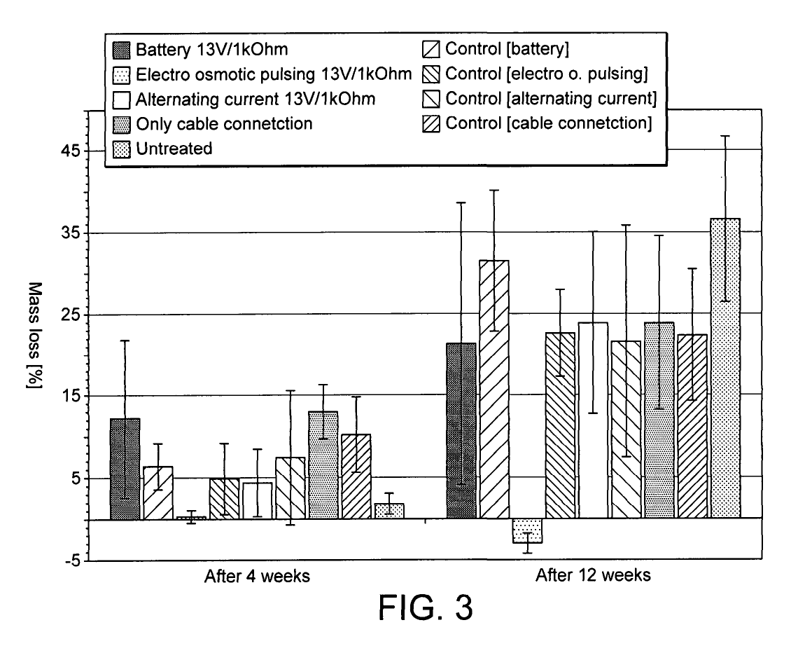

Similar Scots pine sapwood samples were placed in pairs on agar in Petri dishes and exposed to Coniophora puteana (brown rot fungus) as in Example 1. Parts of the samples were removed at 4 and 12 weeks and weighed to determine weight loss as an indicator of fungal damage. In each case, the Petri dishes contained two samples separated by a barrier, with one sample being exposed to the electricity source, a 13V battery, a 13V alternating voltage and a 13V DC electroosmotic pulse source as described above. As further controls were used untreated samples and samples with electrical leads attached but with no voltage applied. The resistance in each case was about 1 kΩ. The results are shown in FIG. 3. As can clearly be seen, while all voltage application provided some protection, the protection afforded by electroosmotic pulsing was significantly superior.

Although this invention has been shown and described with respect to the detailed embodiments thereof, it will be understood by those skilled in the art that various changes in form and detail thereof may be made without departing from the spirit and the scope of the invention.

What is claimed is:

1. A method of inhibiting biological damage to a cellulosic structural element or to an animal feed or bedding material exposed to environmental moisture, said method comprising applying a pulsed asymmetric electrical voltage to a decay-prone region of a said element, feed or material over a prolonged time period.

2. (canceled)

3. The method as claimed in claim 1 wherein said voltage is DC.

4. The method as claimed in claim 3 wherein a pulsed DC voltage is applied.

5. The method as claimed in claim 1 wherein said element is of wood or wood plastic composite.

6. The method as claimed in claim 5 wherein said element is a living tree.

7. The method as claimed in claim 1 wherein said element is cloth.

8. The method as claimed in claim 1 wherein said feed is a pelletized feed.

9. The method as claimed in claim 1 wherein said feed or bedding is hay or straw.

10. The method as claimed in claim 1 wherein said voltage is applied for at least one week.

11. The method as claimed in claim 1 wherein the average modulus voltage and current applied to said element is no more than 50V and 1 mA respectively.

12. The method as claimed in claim 1 for inhibiting termite damage.

13. A cellulosic structural element incorporating apparatus for inhibiting biological damage thereof, said apparatus comprising a source of a pulsed asymmetric electrical voltage electrically connected to a decay-prone region of said element.

14. An apparatus for inhibiting biological damage to a cellulosic structural element, said apparatus comprising a voltage modulator (e.g. a rectifier or transformer) connectable to an electric voltage source and connected via an electric lead to a cellulosic element-engaging electrode.

15. The method as claimed in claim 1 wherein said voltage is applied over a prolonged tie period that is substantially continuous for the operative lifetime of said element.

16. The method as claimed in claim 7 wherein said voltage is applied to wires interwoven within said cloth.

17. The element as claimed in claim 13 wherein the electrical connection comprises electrodes placed on the surface of said element and penetrating into said element.

18. The element as claimed in claim 17 wherein said electrodes are disposed in one or more arrays or grids on a non-exposed side of said element.
